(19) United States
(12) Patent Application Publication
(10) Pub. No.: US 2023/0210624 A1
(43) Pub. Date: Jul. 6, 2023

(54) SURGICAL SHARPS TRAY

(71) Applicant: Sirius Genesis Ltd, London (GB)

(72) Inventors: Umesh Khot, London (GB); Michael Parker, London (GB)

(73) Assignee: Sirius Genesis Ltd, London (GB)

(21) Appl. No.: 18/000,614

(22) PCT Filed: Jun. 4, 2021
(86) PCT No.: PCT/GB2021/051392

§ 371 Date: Dec. 2, 2022

(30) Foreign Application Priority Data
Jun. 4, 2020 (GB) 2008432.3

(51) Int. Cl. A61B 50/33; A61B 50/30; A61B 50/36

(52) U.S. Cl. CPC A61B 50/33; A61B 50/362; A61B 50/3001; A61B 2050/3007; A61B 2050/3008; A61B 2050/3011

(57) ABSTRACT

A receptacle for the safe passing of surgical instruments during surgical procedures, the receptacle comprising: a first pick-up well for receiving one or more surgical instruments and defined by a protective wall and one or more first walls, each of the protective and one or more first walls having a top edge which collectively define an opening to the first pick-up well, and the top edge of the protective wall projecting above the top edges of the one or more first walls; and a handle external to the first pick-up well and comprising an elongate member projecting from the protective wall and adapted to be gripped in a human hand; wherein: the protective wall is located between the elongate member and the first pick-up well; and the elongate member joins the protective wall at a position and orientation such that, in use when a user is gripping the elongate member, the hand of the user does not project above the top edge of the protective wall.

### BACKGROUND OF THE INVENTION

This present disclosure relates to a receptacle for the safe passing of surgical instruments during surgical procedures.

Sharps injuries in the operating theatre are a common occupational hazard. Sharps injuries are typically caused by surgical instruments such as hollow bore needles, suture needles, and scalpels - collectively such instruments are termed “sharps”. Approximately 100,000 sharps injuries occur in UK hospitals each year, making up approximately 17% of all accidents to NHS staff.

Sharps injuries can have serious outcomes. These include transmission of serious infections (such as HIV, Hepatitis B, C and E), and in extreme cases the need for surgery, and sometimes amputation. There can additionally be severe effects on the mental health of health workers who suffer a sharps injury, since not knowing if one has contracted a potentially fatal disease from such an injury can be extremely traumatic.

The problem of sharps injuries in the operating theatres remains largely unaddressed. Whilst protocols can be established with the aim of ensuring the sharps are passed as safely as possible between surgeons and scrub nurses, there is a lack of suitable engineered safety devices (ESDs) which enable sharps to be passed more safely between theatre staff. Currently, sharps such as suture needles, scalpels, surgical scissors, and suture mounted needle holders are passed between scrub nurse and surgeon either directly hand to hand or in a simple container such as a kidney dish. These containers are neither universal nor specifically designed for such tasks. They provide no protection to the fingers of the scrub nurse, especially the thumb and which typically rests along the top edge of the container while the rest of the fingers and palm are holding the under surface. As a result, the scrub nurse is in particular at significant risk of suffering a sharps injury when the surgeon picks up or returns a surgical instrument. Furthermore, the receptacle is flat, making it difficult and fiddly for the surgeon to safely pick up surgical instruments.

In the case of suturing, the current practice is for the scrub nurse mounts the needle with suture material on a needle holder and pass it directly to the surgeon, without making use of a receptacle. The needle holder with the needle can potentially cause a needle stick injury to the assistants or scrub nurse in its path. At return, good practice is to reverse mount the used suture with the sharp end of the needle facing the needle holder shaft. This prevents injury from the sharp end but exposes the surgeon to risk because they must rotate the sharp needle itself in order to achieve this return position.

Alternatives to the use of simple containers such as the kidney dish have been proposed but these have not been successful because they do not address many of the fundamental issues. For example, U.S. Pat. 7,441,655 describes a sharps tray having slots in its base in which surgical instruments can be carried. Cutaways in the walls of the tray enable the surgeon to grasp the surgical instrument lying in the base of the tray. However, the sharps tray still requires the surgeon to place their hands into the bottom of the tray in order to grasp an instrument, and the scrub nurse is still required to grasp the tray around its upper edges, exposing their hand to a potential sharps injury.

U.S. Pat. Application 2019/0307525 describes a medical tray having a recess for receiving a medical instrument. Ribs in the base of the tray are provided so as to space the instrument away from the floor of the tray and facilitate pick up of the instrument. An opening in the wall of the tray is provided to allow longer instruments to project beyond the perimeter of the tray and enable pick up of such instruments without the surgeon being required to place their hand in the tray. However, the sharps tray still requires the surgeon to place their hands into the bottom of the tray in order to grasp smaller instruments, and the scrub nurse is still required to grasp the tray around its upper edges, exposing their hand to a potential sharps injury.

U.S. Pat. Application 2006/0042977 describes a transfer tray for passing surgical instruments. Again, the sharps are placed in the bottom of the tray, with ribs being provided in the floor of the tray so as to space surgical instruments away from the floor of the tray and enable the surgeon to more easily grasp surgical instruments lying in the tray. However, the sharps tray still requires the surgeon to place their hands into the bottom of the tray in order to grasp an instrument. Furthermore, although finger recesses are provided on the underside of the tray so as to allow the scrub nurse to hold the tray from below, these recesses are accessible only once the tray has already been picked up, at which point the nurse has already been exposed to risk by needing to grasp the tray around its edges, placing part of their hand (typically their thumb) within the tray. Switching to holding the tray from beneath also requires the scrub nurse to have both hands available, which is not always possible, and so naturally scrub nurses will in practice often choose to hold the tray by its upper edges.

### SUMMARY OF THE INVENTION

There is provided a receptacle for the safe passing of surgical instruments during surgical procedures, the receptacle comprising:
a first pick-up well for receiving one or more surgical instruments and defined by a protective wall and one or more first walls, each of the protective and one or more first walls having a top edge which collectively define an opening to the first pick-up well, and the top edge of the protective wall projecting above the top edges of the one or more first walls; and
a handle external to the first pick-up well and comprising an elongate member projecting from the protective wall and adapted to be gripped in a human hand; wherein:
   the protective wall is located between the elongate member and the first pick-up well; and
   the elongate member joins the protective wall at a position and orientation such that, in use when a user is gripping the elongate member, the hand of the user does not project above the top edge of the protective wall.

In use, the hand of the user may not project above the top edge of the protective wall when any straight line drawn between the hand and any point on the top edges of the one or more first walls passes through the protective wall.

The receptacle may further comprise a bottom wall joining the bottom edges of the protective and one or more first walls so as to provide a floor for the first pick-up well.

The protective and one or more first walls may each comprise an inner face lying within the first pick-up well, each of the inner faces being raked so as to deflect deposited instruments into the well.

The one or more first walls may include a front second wall which is substantially opposed to the protective wall, the front second wall having a first slot in its top edge for locating a surgical instrument.

The receptacle may be adapted to be supported on a flat surface and the first pick-up well is proportioned such that, when a surgical instrument is deposited in the first pick-up well and located in the first slot, the angle of the surgical instrument to the flat surface is: between 20 and 50 degrees; between 25 and 40 degrees; approximately 30 degrees.

The receptacle may comprise a second pick-up well for receiving one or more surgical instruments, the second pick-up well being of different dimensions to the first pick-up well and being defined by the protective wall and one or more second walls, each of the one or more second walls having a top edge which, along with the top edge of the protective wall, collectively define an opening to the second pick-up well, and the top edge of the protective wall projecting above the top edges of the one or more second walls, the handle being external to the second pick-up well and the protective wall being located between the elongate member and the second pick-up well.

In use, the hand of the user may not project above the top edge of the protective wall when any straight line drawn between the hand and any point on the top edges of the one or more second walls passes through the protective wall.

The receptacle may further comprise a bottom wall joining the bottom edges of the protective and one or more second walls so as to provide a floor for the second pick-up well.

The top edges of the second walls of the second pick-up well may be lower than the top edges of the first walls of the first pick-up well.

The protective and one or more second walls may each comprise an inner face lying within the second pick-up well, each of the inner faces being raked so as to deflect deposit instruments into the second pick-up well.

The first and second pick-up wells may be adjacent to one another and a second wall of the first pick-up well is shared in common with a third wall of the second pick-up well.

The protective wall and/or one or more of the first/second walls may comprise lower edges adapted to support the receptacle when the receptacle is placed on a flat surface.

The lower edges of the protective and/or first/second walls may be one and the same as the bottom edges of the first walls.

The lower edges of the protective and/or first/second walls may comprise one or more skirts configured to support the receptacle when placed on a flat surface.

The one or more second walls may include a front third wall which is substantially opposed to the protective wall, the front third wall having a second slot in its top edge for locating a surgical instrument.

The receptacle may be adapted to be supported on a flat surface and the second pick-up well is proportioned such that, when a surgical instrument is deposited in the second pick-up well and located in the second slot, the angle of the surgical instrument to the flat surface is: between 20 and 50 degrees; between 25 and 40 degrees; approximately 30 degrees.

The elongate member joins the protective wall at a position and orientation such that, when the receptable is placed on a flat surface, the elongate member may be sufficiently spaced apart from that flat surface so as to permit the fingers of a user to grip the elongate member.

The receptacle may further comprise a receiving well for receiving surgical waste and defined by a plurality of third walls, each of the plurality of third walls having a top edge which collectively define an opening to the receiving well.

The receiving well may be spaced apart from the protective wall by the first and/or second pick-up wells, and the plurality of third walls includes the front first wall and/or front second wall.

The receptacle may further comprise a needle well for receiving a surgical needle, the needle well being spaced apart from the first and/or second pick-up wells by the receiving well.

The needle well may be substantially cylindrical.

The plurality of third walls may include a front third wall which is substantially opposed to the front first wall and/or front second wall, the front third wall comprising the needle well.

The distance between the protective wall and the front third wall may be less than the distance between the protective wall and the front second wall, and the needle well is adjacent to the second pick-up well but spaced apart by the receiving well.

The protective wall may comprise one or more sliding or folding portions which, when extended, provide the top edge of the protective wall.

The receptacle may be moulded as a single piece.

The walls of the receptacle may be substantially hollow and adapted such that the receptacle is stackable with a plurality of other receptacles.

### BRIEF DESCRIPTION OF THE DRAWINGS

The present invention is described by way of example with reference to the accompanying drawings. In the drawings:

FIG. 1 is a front perspective view of a surgical sharps tray configured in accordance with the principles described herein.

FIG. 2 is a rear perspective view of a surgical sharps tray configured in accordance with the principles described herein.

FIG. 3 is a side view of a surgical sharps tray configured in accordance with the principles described herein.

FIG. 4 is a bottom perspective view of a surgical sharps tray configured in accordance with the principles described herein.

FIG. 5 is a photograph illustrating the position of sharp surgical instruments in a surgical sharps tray configured in accordance with the principles described herein.

FIG. 6 illustrates a position of a surgical sharp in a pick-up well of a surgical sharps tray configured in accordance with the principles described herein.

FIG. 7 is a front perspective view of a surgical sharps tray configured in accordance with the principles described herein.

### DETAILED DESCRIPTION OF THE INVENTION

The following description is presented by way of example to enable a person skilled in the art to make and use the invention. The present invention is not limited to the embodiments described herein and various modifications to the disclosed embodiments will be apparent to those skilled in the art.

The present disclosure relates to an improved receptacle for the safe passing of surgical sharps. Such a receptacle may be referred to herein as a surgical sharps tray. In particular, a sharps tray configured in accordance with the principles described herein provides hand protection for the person holding the tray (e.g. the scrub nurse) and enables the surgeon to safely pick up a variety of sharp surgical instruments without needing to place their hand into a container holding the sharp instrument.

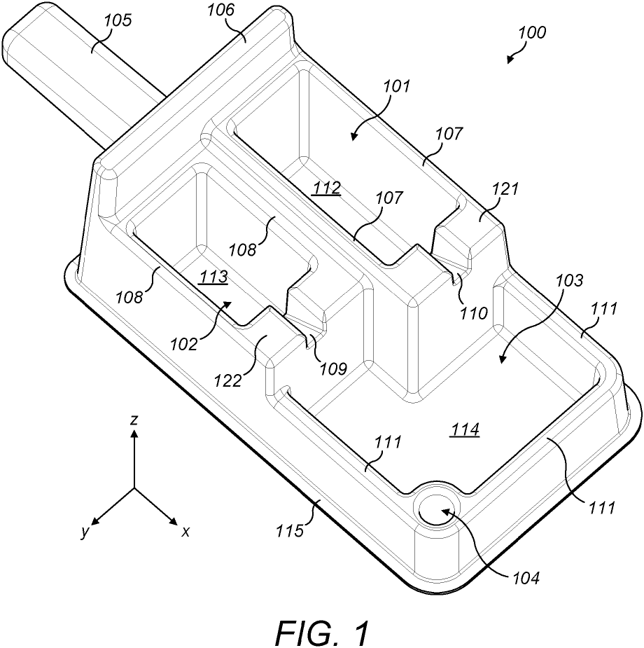

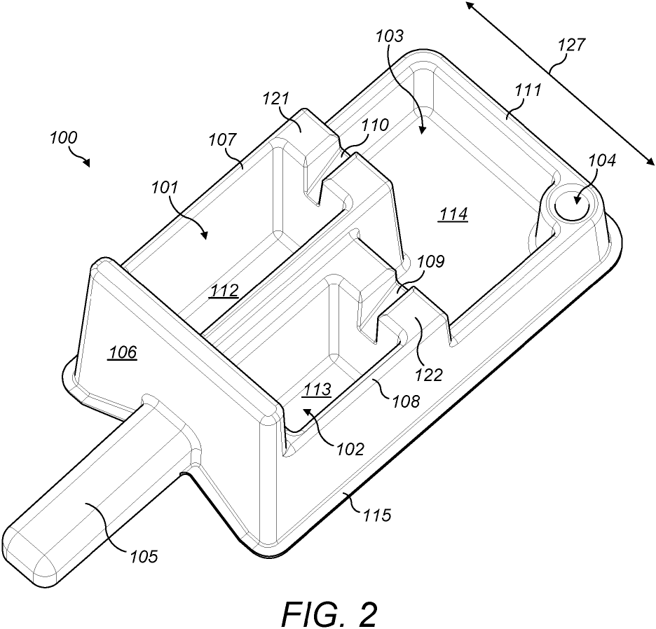

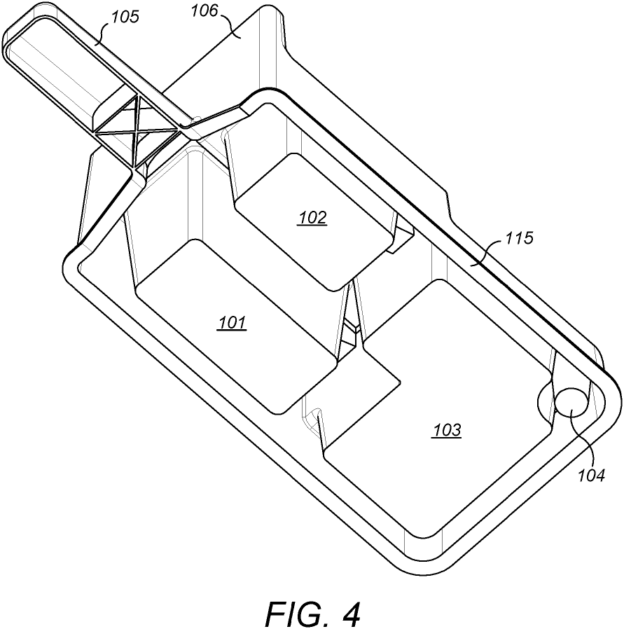

An exemplary surgical sharps tray 100 configured in accordance with the principles described herein is shown in FIGS. 1 to 4. The sharps tray 100 includes a first pick-up well 101, a second pick-up well 102, and a receiving well 103. In general the sharps tray may comprise one or more pick-up wells. The sharps tray may further comprise a needle well 104. The base of the sharps tray (e.g. as defined by the bottom edges of its walls) is adapted such that the sharps tray is stable when placed on a flat surface such as the top of a surgical trolley. Skirts 115 may be provided to improve the stability of the sharps tray.

A protective wall 106 is provided between the one or more pick-up wells and a handle 105 is provided to allow the sharps tray to be safely carried. Each of the one or more pick-up wells is defined between the protective wall and a plurality of other walls so as to form a cavity into which surgical instruments may be placed. A floor is provided to close off the bottom of each well and prevent instruments falling through the wells. The one or more wells are substantially open at their tops so as to provide an opening through which surgical instruments may be received into the wells.

For example, in the figures, the first pick-up well 101 is defined by protective wall 106, side walls 107, front wall 121, and floor 112; and the second well 102 is defined by protective wall 106, side walls 108, front wall 122, and floor 113. The front wall of each pick-up well substantially opposes the protective wall, with the side walls connecting the front wall to the protective wall. In the example in the figures, for each pick-up well, the front and protective walls are parallel to one another, and the side walls are parallel to one another, so as to define a cuboid space into which surgical instruments may be held. More generally the pick-up wells may be defined by the protective wall and any number of other walls arranged to form a suitable cavity for receiving surgical instruments - for example, a single curved wall may be provided which is joined at both ends to the protective wall, the instrument cavity being defined between the protective and curved walls. In general, the space defined within a pick-up well may have any shape - for example, the space may be, substantially, a cuboid, a sphere or part-sphere, a cylinder, triangular, etc.

One or more side walls of adjacent pick-up wells may be at least partially one and the same wall - for example, the side wall 107 lying between pick-up wells 101 and 102 may be considered to be one and the same as the side wall 108 lying between the pick-up wells. In some examples, the floor of a pick-up well may be one and the same as one or more walls defining the well - for instance, at least the lower parts of one or more walls may comprise a curved section which meets a lower part of the protective wall so as to create a “floor” to the pick-up well, though the floor may not be distinct from those one or more walls.

Each of the pick-up wells may comprise a slot in the wall of the pick-up well substantially opposing the protective wall 106 - e.g. slot 110 in front wall 121 of pick-up well 101, or slot 109 in front wall 122 of pick-up well 102. The slot is adapted to locate a surgical instrument safely within the pick-up well and prevent the instrument moving around as the sharps tray is moved about the operating theatre. For example, the slot may be sized so as to accommodate one or more of: scalpels, needle holders, surgical syringes, and surgical scissors. In surgical trays with a plurality of pick-up wells, different pick-up wells could have slots of different sizes so as to optimally accommodate surgical instruments of different sizes. A slit with a width of approximately 10-20 mm has been found to suitably locate a wide range of surgical instruments in a pick-up well and improve safety by preventing the sharp instrument from moving/rolling around the top of the walls of the pick-up well. Slots (e.g. 109 or 110) in the pick-up wells may be U-shaped - e.g. the slots may have a rounded base in which a surgical instrument can rest.

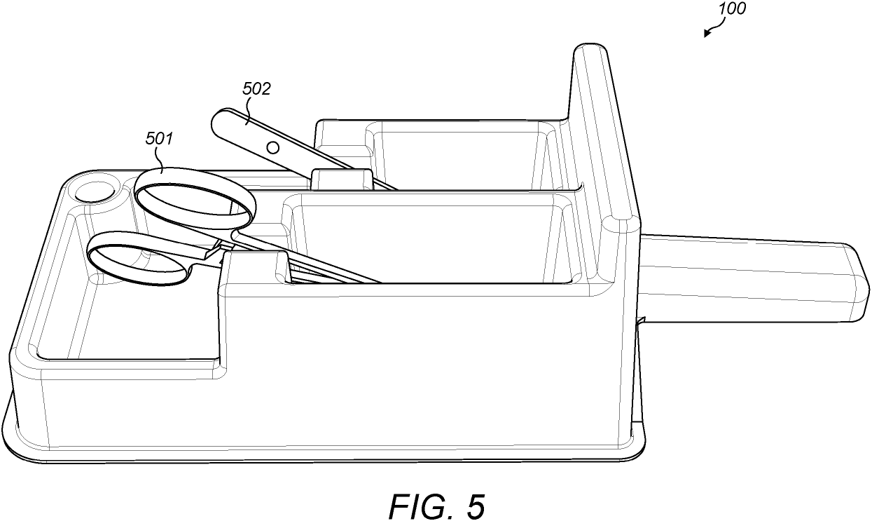

Each of the one or more pick-up wells are provided to receive sharp surgical instruments with their sharp ends pointing downwards into the bottom of the well at the base of the protective wall. This is illustrated in FIG. 5 which shows a needle holder 501 and a scalpel 502 resting in the pick-up wells of sharps tray 100.

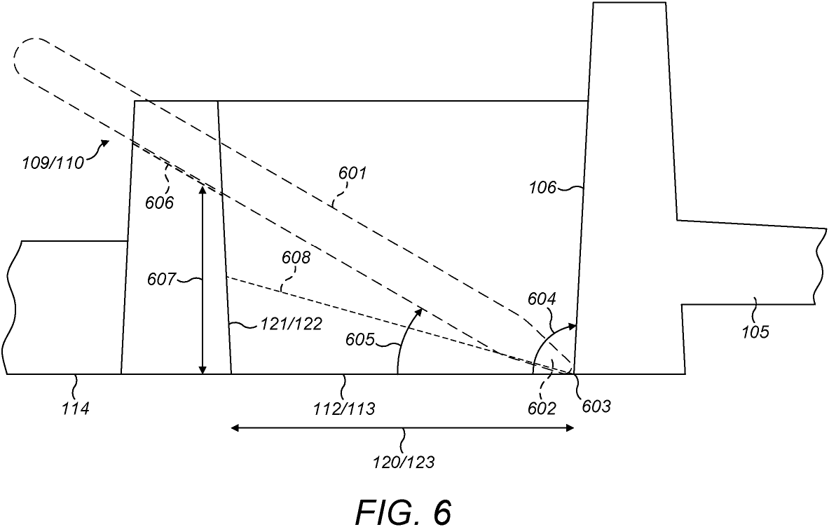

The position of surgical sharps in a pick-up well is further illustrated in FIG. 6. An exemplary sharp instrument 601 (e.g. a scalpel) is illustrated in-situ in a pick-up well. The instrument 601 is located in the slot 109/110 of the front wall 121/122. The sharp end 602 of the instrument 601 is located at the base of the protective wall 106. Preferably the protective wall 106 meets the floor 112/113 of the pick-up well at an angle 604 which is one or more of: less than 150 degrees, less than 120 degrees, less than 100 degrees, and approximately 90 degrees. Such an arrangement helps to ensure that the sharp end 602 of the instrument remains safely located at the base of the pick-up well because, should the instrument move about and the sharp end become displaced in the pick-up well, the relative orientations of the protective wall and floor will act to re-locate the sharp end of the instrument in the join 603 between the protective wall and floor. Preferably the base of the protective wall 106 intersects the floor 112/113 of the pick-up well along an abrupt transition line 603 so as to provide a well-defined crease for locating the sharp end of the instruments - for example, the radius of curvature of a curved join between the protective wall and floor may be one or more of: less than 20 mm; less than 10mmm; less than 5 mm.

The length of the pick-up well 120/123 (along the x-direction in FIG. 1) is selected such that, when located in the pick-up well, the surgical instruments for which it is intended to be used project out of the pick-up well in order to facilitate easy pick up of the instrument by, for example, the surgeon. Since the sharp end of the instrument is for location in the base of the pick-up well, the non-sharp end of the instrument projects out of the well for safe pick up, without the surgeon being required to put their hand into the pick-up well to retrieve the instrument. In examples in which a sharps tray includes more than one pick-up well, each well is preferably of different dimensions so as to optimally accommodate surgical instruments of different sizes. For example, in FIGS. 1-4 the longer pick-up well 101 may be provided for longer instruments such as needle holders, and the shorter pick-up well 102 may be provided for shorter instruments such as scalpels.

The dimensions of the pick-up wells are selected such that surgical instruments received into the pick-up well and supported at a wall of the pick-up well opposing the protective wall (e.g. supported in a slot 109/110) are provided at an angle to the horizontal when the surgical tray is supported at a horizontal surface and/or when held substantially horizontally by handle 105 (e.g. by a scrub nurse). For example, in FIG. 6 the length 120/123 (along the x-direction in FIG. 1) of the pick-up well and the height 607 (along the z-direction in FIG. 1) of the slot 109/110 in the front wall 121/122 (relative to the base of the protective wall, i.e. 603) are selected such that the angle 605 of the longitudinal axis of a surgical instrument 601 in the pick-up well to the horizontal when the tray is placed on a horizontal surface is one or more of: at least 20 degrees; at least 25 degrees; approximately 30 degrees; between 20 and 45 degrees.

The inventors have recognised that, by providing the surgical instruments at an angle to the horizontal, the weight of the surgical instrument helps to locate the sharp end of the instrument in the base of the pick up well and prevent the instrument moving about. This is an improvement over conventional sharps trays which provide surgical instruments flat in the bottom of a tray where they are prone to sliding about. Furthermore, by providing the surgical instruments at an angle it is possible to allow more of a surgical instrument to project out of the pick-up well without the instrument becoming unstable in the well. This provides for easier pick up by the surgeon and enables the length of the pick-up well to be substantially shorter than conventional sharps trays and hence even relatively short instruments such as small scalpels may be accommodated in the pick-up well because even such instruments will project out of the well.

If the wall of the pick-up well on which the non-sharp end of the surgical instrument rests has a non-negligible thickness, the base 606 of a slot 109/110 provided to locate a surgical instrument 601 in the pick-up well is preferably commensurate with angle 605 so that the rake of the base of the slot substantially matches the angle of a surgical instrument is provided in the well.

In the examples given herein, the floor of a pick-up well is substantially horizontal when the sharps tray is placed on a horizontal surface. However, more generally the floor of the pick-up well may itself be at an angle to the horizontal and/or may not be planar in shape. For example, it will be appreciated that the floor of a pick-up well could slope upwards towards the front wall (e.g. as illustrated by dotted line 608 in FIG. 6) without affecting the functionality of the pick-up well.

Whilst some conventional sharps trays include features designed to increase the safety for a surgeon picking up an instrument from the tray, those sharps trays do not reduce the risk of a sharps injury for the scrubs nurse (or other theatre staff) who must hold the sharps tray whilst the surgeon is picking up instruments from the tray or replacing instruments in the tray. This is to some extent a consequence on prior designs of sharp trays focussing on minimising the risks to the surgeon at the expense of the risks to the scrub nurse. This is despite scrub nurses and other assistants suffering from a large proportion of the sharps injuries experienced in operating theatres. The sharps tray may be considered to be a form of personal protective equipment (PPE) whose use can significantly reduce the risk of sharps injuries in operating theatres to both the surgeons and the scrub nurses and other surgical assistants.

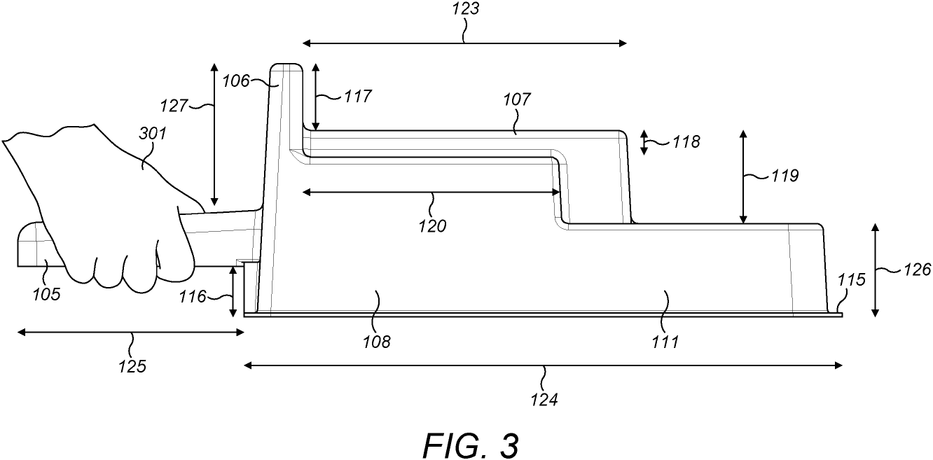

In sharps trays configured in accordance with the principles described herein, the protective wall 106 projects above the height of the other walls of the pick-up wells, as illustrated by dimension 117 in FIG. 3. Furthermore, the handle joins the back side of the protective wall at a position and orientation such that, in use when a user is gripping the elongate member, the hand of the user does not project above the top edge of the protective wall. This arrangement ensures that the hand of the holder of the sharps tray is not exposed to the sharps which are placed into or picked up from the one or more pick-up wells of the tray.

In order to minimise the risk of sharps injuries, the top of the handle 105 is preferably at least 50 mm, and most preferably at least 55 mm, below the top of the protective wall 106, as indicated by the dimension 127 in FIG. 3. The top of protective wall 106 is preferably at least 20 mm, and most preferably at least 25 mm, above the height of the top of the opposing wall of the pick-up well (e.g. its front wall 121/122 in the figures) so as to protect the holder of the sharps tray against inaccurate placement of the sharps in the well by the surgeon. For each pick-up well, it is the height of the front wall opposing the protective wall relative to the height of the protective wall which is important because the height of the front wall determines the height to which the surgeon must lift the sharp in order to place it in the well. The height of the front wall is the height of its top edge ignoring any slot (e.g. 109/110) provided in its top edge. In some examples, the height of protective wall may not be uniform over the width of the tray - in such cases, either the average height of the protective wall or the height of the protective wall at the point opposing the entry point of surgical instruments at the front wall may be taken as the height of the protective wall for the purposes of determining the relative heights of the protective wall and the opposing wall of a pick-up well.

The handle 105 may be any suitable elongate member suitable to be gripped by a human hand so as to permit theatre staff to carry the sharps tray and without requiring any part of their hands to lie within the one or more pick-up wells of the tray. FIG. 3 illustrates the typical grip of the hand 301 of a scrub nurse when holding the handle of the sharps tray. The hand 301 may be considered to not project above the top edge of the protective wall when any straight line drawn between the hand and any point on the top edges of the one or more first walls passes through the protective wall. The handle is positioned relative to the top of the protective wall and oriented such that in use the hand of the holder does not project above the top edge of the protective wall. The hand may be considered to include those parts of the body lying between the fingertips and the distal ends of the radius and ulnar bones.

In the examples shown in FIGS. 1-4 the handle is substantially orthogonal to the back side of the protective wall 106. In other examples, the handle may be at any other angle to the protective wall. For example, the handle may comprise an elongate member connected at one end to the protective wall but at an acute angle to the wall. In some examples, the handle comprise an elongate member which is approximately parallel to the protective wall and connected to the wall by one or more connecting members which permit the member to be grasped by the human hand.

It is advantageous if the handle is spaced apart from the base of the sharps tray such that, when the sharps tray is supported on a flat surface, the elongate member of the handle may be grasped by a hand without first lifting the tray from the surface by other means, typically by gripping the walls of the pick up wells which would introduce an increased risk of a sharps injury. In FIG. 3 it can be observed that the handle 105 is spaced apart from the base of the tray by distance 116.

The protective wall 106 may be substantially thicker (in the x direction) than the other walls defining the one or more pick-up wells, so as to distance the hand of a user gripping the handle from the pick-up wells and the sharps contained therein. For example, the protective wall may be at least 20 mm thick.

The sharps tray may further comprise a receiving well 103 for receiving non-sharp surgical waste, such as used swabs and other surgical materials. The receiving well is defined between substantially lower walls 111 than those of the one or more pick-up wells 101/102 and includes a floor 114 which may or may not be provided by the lower parts of the walls 111. For example, the height of the walls of the receiving well may be approximately 50% of the height of the walls of a pick-up well. As well as provided a convenient receptacle for used surgical materials during surgery, the receiving well ensures that sharp instruments located in the one or more pick-up wells do not project beyond the perimeter of the sharps tray. For example, the receiving well may be of a length such that, for the surgical instruments which it is envisaged will be received into a pick-up well, those surgical instruments will, when located in the pick-up well, overhang the receiving well but will not extend beyond the lateral bounds of the sharps tray (i.e. in the x and y directions defined in FIG. 1). This reduces the risk that a surgical instrument could be knocked out of a pick-up well by theatre staff because: (a) other theatre staff are more likely to notice the sharps tray than the end of a potentially thin surgical tool poking out of a pick-up well and so will give the sharps tray more space; (b) since scrub nurses will adapt to the size of the tray, the holder of the sharps tray will find it easier to judge how much space is required to safely manoeuvre the sharps tray into place adjacent to a surgeon without knocking the ends of the surgical tools poking out of a pick-up well; and (c) if the sharps tray does collide with theatre staff or equipment, the sharps are less likely to be knocked out of the pick-up wells since the tray itself is more likely to receive the brunt of the collision.

The sharps tray may further comprise one or more needle wells 104 from which a surgeon may pick up a needle (typically a straight needle) or into which a used needle may be received from a surgeon. The needle well may be defined in a wall of the receiving well 103 and/or may be defined between walls of a height substantially lower than (e.g. approximately 50% lower) than those of a pick-up well. The depth of the needle well is selected appropriate to the length of surgical needles - for example, the needle well may have a depth of approximately 30-35 mm. It is advantageous for the needle well to be spaced apart from the pick-up wells by the receiving well (e.g. as shown in FIGS. 1-4) so as to minimise the risk that a surgeon picking or returning a needle to the needle well knocks a surgical instrument located in one of the pick-up wells. In a sharps tray having a plurality of pick-up wells of different sizes, the needle well is preferably located adjacent such that the shortest straight line between the needle well and the protective wall passes through the shortest of the pick-up wells, as is the case in the exemplary sharps tray illustrated in FIGS. 1-4.

In the example shown in FIGS. 1-4, the length 124 of the sharps tray not including the handle is approximately 214 mm, the length 125 of the handle 105 is approximately 90 mm, the height 126 of the walls of the receiving well (and its depth) is approximately 35 mm, the height 119 of the walls of the first pick-up well over the height of the walls of the receiving well is approximately 35 mm, and the height 117 of the protective wall over the height of the walls of the first pick-up well is approximately 25 mm. The length 123 of the first pick-up well 101 is approximately 120 mm and its depth is approximately 70 mm. The length 120 of the second pick-up well 102 is approximately 100 mm and its depth is approximately 60 mm. The height 118 of the walls of the first pick-up well 101 over the height of the walls of the second pick-up well 102 is approximately 10 mm. The width of the sharps tray 127 is approximately 120 mm.

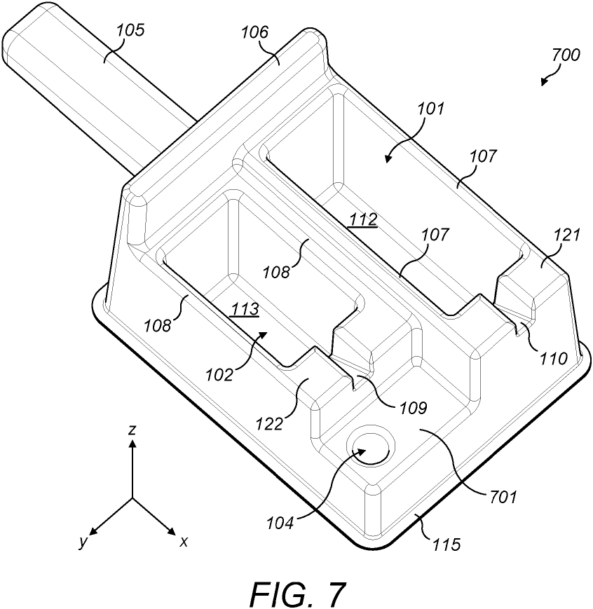

A receiving well may not be included in the sharps tray. In the example shown in FIG. 7, the length of the sharps tray not including the handle is approximately 170 mm, the length of the handle 105 is approximately 104 mm, the height of the protective wall 106 over the height of the walls of the first pick-up well 101 is approximately 30 mm. The height of the protective wall 106 over the handle 105 is approximately 50 mm. The total height of the protective wall above the base of the sharps tray (e.g. skirt 115) is approximately 95 mm. The internal length of the first pick-up well 101 is approximately 115 mm and its depth is approximately 70 mm. The internal length of the second pick-up well 102 is approximately 75 mm and its depth is approximately 60 mm. The height of the walls of the first pick-up well 101 over the height of the walls of the second pick-up well 102 is approximately 10 mm. The width of the sharps tray 127 is approximately 120 mm. The height of the flat front portion 701 in which the needle well 104 is located is approximately 30 mm below the height of the walls of the first pick-up well 101. The diameter of the needle well is approximately 15 mm. The width of the protective wall 106 (i.e. in the x-direction indicated in FIG. 7) is approximately 10 mm.

In some examples, the protective wall of a sharps tray configured in accordance with the principles described herein may comprise one or more folding or sliding pieces which are configured to, when extended, increase the height of the protective wall and/or extend over the handle so as to further shield the hand of a user holding the handle of the sharps tray.

A sharps tray configured in accordance with the principles described herein may be fabricated in any suitable manner. The sharps tray may be moulded, e.g. from plastic or a paper pulp. The sharps tray may be thermoformed from a plastic sheet: this approach is low cost and results in a lightweight product since all the surfaces of the tray are formed of the substantially uniform thickness sheet and so the walls defining the various wells of the sharps tray would typically be hollow in order to achieve the desired thickness. The sheet defining the outer surface of a wall may be provided with skirts 115 so as to maximise the stability of the sharps tray when placed on a flat surface, such as the top of a surgical trolley.

It is advantageous if the surfaces of the sharps tray are configured such that sharps trays may be stacked on top of one another. This can be achieved by arranging that all of the approximately vertical surfaces are slightly raked. This has the further advantage that the walls defining each pick-up well naturally deflect sharp instruments into the base of the wells, safely securing the sharp ends away from surgical staff.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein. In view of the foregoing description it will be evident to a person skilled in the art that various modifications may be made within the scope of the invention.

1. A receptacle for the safe passing of surgical instruments during surgical procedures, the receptacle comprising:
a first pick-up well for receiving one or more surgical instruments and defined by a protective wall and one or more first walls, each of the protective and one or more first walls having a top edge which collectively define an opening to the first pick-up well, and the top edge of the protective wall projecting above the top edges of the one or more first walls; and
a handle external to the first pick-up well and comprising an elongate member projecting from the protective wall and adapted to be gripped in a human hand; wherein:
the protective wall is located between the elongate member and the first pick-up well; and
the elongate member joins the protective wall at a position and orientation such that, in use when a user is gripping the elongate member, the hand of the user does not project above the top edge of the protective wall.

2. A receptacle as claimed in claim 1, wherein, in use, the hand of the user does not project above the top edge of the protective wall when any straight line drawn between the hand and any point on the top edges of the one or more first walls passes through the protective wall.

3. A receptacle as claimed in claim 1, further comprising a bottom wall joining the bottom edges of the protective and one or more first walls so as to provide a floor for the first pick-up well.

4. A receptacle as claimed in claim 1, wherein the protective and one or more first walls each comprise an inner face lying within the first pick-up well, each of the inner faces being raked so as to deflect deposited instruments into the well.

5. A receptacle as claimed in claim 1, wherein the one or more first walls include a front second wall which is substantially opposed to the protective wall, the front second wall having a first slot in its top edge for locating a surgical instrument.

6. A receptacle as claimed in claim 5, wherein the receptacle is adapted to be supported on a flat surface and the first pick-up well is proportioned such that, when a surgical instrument is deposited in the first pick-up well and located in the first slot, the angle of the surgical instrument to the flat surface is: between 20 and 50 degrees; between 25 and 40 degrees; approximately 30 degrees.

7. A receptacle as claimed in claim 1, wherein the receptacle comprises a second pick-up well for receiving one or more surgical instruments, the second pick-up well being of different dimensions to the first pick-up well and being defined by the protective wall and one or more second walls, each of the one or more second walls having a top edge which, along with the top edge of the protective wall, collectively define an opening to the second pick-up well, and the top edge of the protective wall projecting above the top edges of the one or more second walls, the handle being external to the second pick-up well and the protective wall being located between the elongate member and the second pick-up well.

8. A receptacle as claimed in claim 7, wherein, in use, the hand of the user does not project above the top edge of the protective wall when any straight line drawn between the hand and any point on the top edges of the one or more second walls passes through the protective wall.

9. A receptacle as claimed in claim 7, further comprising a bottom wall joining the bottom edges of the protective and one or more second walls so as to provide a floor for the second pick-up well, and/or
wherein the top edges of the second walls of the second pick-up well are lower than the top edges of the first walls of the first pick-up well.

10. (canceled)

11. A receptacle as claimed in claim 7 to 10, wherein the protective and one or more second walls each comprise an inner face lying within the second pick-up well, each of the inner faces being raked so as to deflect deposit instruments into the second pick-up well.

12. A receptacle as claimed in claim 7, wherein the first and second pick-up wells are adjacent to one another and a second wall of the first pick-up well is shared in common with a third wall of the second pick-up well.

13. (canceled)

14. (canceled)

15. (canceled)

16. A receptacle as claimed in claim 7 to 15, wherein the one or more second walls include a front third wall which is substantially opposed to the protective wall, the front third wall having a second slot in its top edge for locating a surgical instrument.

17. A receptacle as claimed in claim 16, wherein the receptacle is adapted to be supported on a flat surface and the second pick-up well is proportioned such that, when a surgical instrument is deposited in the second pick-up well and located in the second slot, the angle of the surgical instrument to the flat surface is: between 20 and 50 degrees; between 25 and 40 degrees; approximately 30 degrees.

18. A receptacle as claimed in claim 1, wherein the elongate member joins the protective wall at a position and orientation such that, when the receptable is placed on a flat surface, the elongate member is sufficiently spaced apart from that flat surface so as to permit the fingers of a user to grip the elongate member.

19. A receptacle as claimed in claim 1, further comprising a receiving well for receiving surgical waste and defined by a plurality of third walls, each of the plurality of third walls having a top edge which collectively define an opening to the receiving well.

20. (canceled)

21. A receptacle as claimed in claim 19, further comprising a needle well for receiving a surgical needle, the needle well being spaced apart from the first and/or second pick-up wells by the receiving well;
wherein the needle well is substantially cylindrical.

22. (canceled)

23. A receptacle as claimed in claim 21, wherein the plurality of third walls includes a front third wall which is substantially opposed to the front first wall and/or front second wall, the front third wall comprising the needle well.

24. A receptacle as claimed in claim 23, wherein the distance between the protective wall and the front third wall is less than the distance between the protective wall and the front second wall, and the needle well is adjacent to the second pick-up well but spaced apart by the receiving well.

25. A receptacle as claimed in claim 1, wherein the protective wall comprises one or more sliding or folding portions which, when extended, provide the top edge of the protective wall.

26. (canceled)

27. A receptacle as claimed in claim 1, wherein the walls of the receptacle are substantially hollow and adapted such that the receptacle is stackable with a plurality of other receptacles.
